(19) United States
(12) Patent Application Publication
(10) Pub. No.: US 2024/0366371 A1
(43) Pub. Date: Nov. 7, 2024

(54) OUTER COVERING WITH PROTRUDING SEALING MEMBERS FOR A PROSTHETIC HEART VALVE

(71) Applicant: EDWARDS LIFESCIENCES CORPORATION, Irvine, CA (US)

(72) Inventors: Michael Bukin, Pardes Hanna (IL); Giolnara Pinhas, Hadera (IL); Elena Sherman, Pardes Hana (IL); Nikolai Gurovich, Hadera (IL)

(21) Appl. No.: 18/777,392

(22) Filed: Jul. 18, 2024

Related U.S. Application Data
(63) Continuation of application No. PCT/US2023/011337, filed on Jan. 23, 2023.
(60) Provisional application No. 63/267,382, filed on Jan. 31, 2022.

(51) Int. Cl. A61F 2/24

(52) U.S. Cl. CPC A61F 2/2418; A61F 2/243; A61F 2/2436; A61F 2220/0075

(57) ABSTRACT

Sealing members for an outer skirt of a prosthetic heart valve are disclosed. As one example, a prosthetic heart valve can include an annular frame, a plurality of leaflets arranged within an interior of the frame, and an outer skirt disposed around an outer surface of the frame. The outer skirt can comprise at least one folded portion and stitches extending through the folded portion to form a radially protruding rib.

### CROSS-REFERENCE TO RELATED APPLICATION

This application is a continuation of PCT application no. PCT/US2023/011337, filed Jan. 23, 2023, which claims the benefit of U.S. Provisional Patent Application No. 63/267,382, filed Jan. 31, 2022, the entire disclosure of each of which is incorporated herein by reference.

### FIELD

The present disclosure relates to prosthetic heart valves, and in particular to outer coverings or skirts with protruding sealing members for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a cylindrical metal frame or stent and prosthetic leaflets mounted inside the frame. These valves can also include one or more coverings (or skirts) spanning a circumference of the frame, on an inner or outer surface of the frame. These coverings can be configured to establish a seal with the native tissue when the prosthetic heart valve is placed at the implantation site (and thus may be referred to as sealing members). However, the native tissue (e.g., at the native valve annulus or arterial wall around the native valve) can have an irregular shape while the frame of the prosthetic heart valve is generally cylindrical. As a result, gaps can be formed between the prosthetic heart valve and native heart valve when the prosthetic heart valve is implanted within the native heart valve, even when coverings are included on the prosthetic heart valve.

### SUMMARY

Described herein are prosthetic heart valves, delivery apparatuses, and methods for implanting prosthetic heart valves. In particular, described herein are examples of coverings for a prosthetic heart valve and methods of making and using such coverings. Prosthetic heart valves can include a frame and a leaflet assembly arranged on an inner surface of the frame. The prosthetic heart valve can include a covering arranged around a circumference of the frame and on an outer surface of the frame. The covering can include one or more sealing members that protrude radially outward and away from the frame. In some examples, the one or more sealing members can extend circumferentially around the frame. The one or more sealing members can be configured such that the prosthetic heart valve, once implanted, better conforms to a shape of the surrounding native tissue. As such, the coverings and prosthetic heart valves disclosed herein can, among other things, overcome one or more of the deficiencies of typical prosthetic heart valves.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can comprise a frame, a valve structure arranged within an interior of the frame, and an outer skirt or sealing member disposed around an outer surface of the frame, the outer skirt comprising one or more sealing members that protrude radially outward and away from the frame and that extend circumferentially around the frame.

In some examples, a prosthetic heart valve comprises: an annular frame; a plurality of leaflets arranged within an interior of the frame; and an outer skirt disposed around an outer surface of the frame, where the outer skirt comprises at least one folded portion and stitches extending through the folded portion to form a radially protruding rib.

In some examples, a prosthetic heart valve comprises: an annular frame comprising a plurality of interconnected struts, and an outer skirt disposed around an outer surface of the frame. The outer skirt comprises an inner surface disposed against the outer surface of the frame and an outer surface arranged opposite the inner surface; a circumferentially extending first row of first apertures and a circumferentially extending second row of second apertures; a plurality of stitches formed between the first and second rows, each stitch of the plurality of stitches extending between a respective one of the first apertures and a respective one of the second apertures; and a folded portion of the outer skirt between the first and second rows of apertures that forms a rib that protrudes radially outward from the outer surface of the outer skirt and away from the frame.

In some examples, a prosthetic heart valve comprises: an annular frame comprising a plurality of interconnected struts; a plurality of leaflets arranged within an interior of the frame; and an outer skirt disposed around an outer surface of the frame. The outer skirt comprises an inflow edge portion secured to an inflow end of the frame; an outflow edge portion secured to a first portion of the plurality of interconnected struts; opposing inner and outer surfaces, where the inner surface faces and is disposed against the outer surface of the frame; and a sealing member that spirals around a circumference of the outer skirt, between the inflow edge portion and toward the outflow edge portion. The sealing member comprises a folded portion of the outer skirt that extends radially outward from the outer surface of the outer skirt and away from the frame.

In some examples, a prosthetic heart valve comprises: a radially expandable and compressible annular frame; a plurality of leaflets arranged within an interior of the frame; and an outer skirt disposed around an outer surface of the frame, the outer skirt comprising one or more folded portions forming one or more radially outwardly protruding ribs that remain folded when the prosthetic heart valve is in a radially compressed state and a radially expanded state.

In some examples, a prosthetic heart valve comprises one or more of the components recited in Examples 1-73 below.

In some examples, a method of assembling a prosthetic heart valve comprises: securing a fabric skirt around an outer surface of a frame of a prosthetic heart valve; extending a suture in an alternating pattern between first apertures of a first row of spaced apart first apertures in the skirt and second apertures of a second row of spaced apart second apertures in the skirt, the first and second rows extending circumferentially along the skirt and spaced apart from one another in an axial direction; and tightening the suture to form a protruding rib between the first and second rows, the protruding rib comprising a folded portion of the skirt that protrudes radially outward from a remainder of the skirt that is disposed against the frame.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2 is a side elevation view of a delivery apparatus for a prosthetic device, such as a prosthetic valve, according to an example.

FIG. 3A is a side view of an outer skirt for a prosthetic heart valve, according to one example, the outer skirt shown in a flattened configuration.

FIG. 3B is a side view of an outer skirt for a prosthetic heart valve, according to another example, the outer skirt shown in a flattened configuration and comprising a plurality of rows of apertures.

FIG. 4A is a perspective view of the outer skirt of FIG. 3B disposed around an exemplary frame of a prosthetic heart valve, the outer skirt comprising a radially outwardly protruding sealing member configured as a spiraling rib.

FIG. 4B is another perspective view of the outer skirt disposed around the exemplary frame of FIG. 4A.

FIG. 5A is a perspective view of the outer skirt of FIG. 3B secured around an exemplary frame of a prosthetic heart valve.

FIG. 5B is a perspective view of the outer skirt disposed around the exemplary frame of FIG. 5A and a portion of a method of forming the spiraling rib of FIGS. 4A-4B, including extending a suture through adjacent rows of apertures in the outer skirt.

FIG. 5C is a perspective view of the outer skirt disposed around the exemplary frame of FIG. 5A with the spiraling rib formed with two revolutions around the outer skirt and frame.

FIG. 5D is another perspective view of the spiraling rib extending radially outward from the outer skirt of FIG. 5C.

FIG. 5E is yet another perspective view of the spiraling rib extending radially outward from the outer skirt of FIG. 5C.

FIG. 6A is a perspective view of an outer skirt disposed around a frame of a prosthetic heart valve, the outer skirt comprising a radially outwardly protruding sealing member configured as a spiraling rib having a single revolution around the outer skirt and frame.

FIG. 6B is another perspective view of the outer skirt and single-revolution spiraling rib of FIG. 6A.

FIG. 7A is a perspective view of an outer skirt disposed around a frame of the prosthetic heart valve with a single, radially protruding circular rib formed in the outer skirt.

FIG. 7B is another perspective view of the outer skirt and circular rib on FIG. 7A.

FIG. 8A is a perspective view of an outer skirt disposed around a frame of the prosthetic heart valve depicting a method of forming a plurality of circular ribs in the outer skirt by utilizing a plurality of sutures extending through a plurality of rows of apertures in the outer skirt.

FIG. 8B is a perspective view of the outer skirt of FIG. 8A with multiple circular ribs formed in the outer skirt.

FIG. 8C is another perspective view of the outer skirt of FIG. 8A showing the multiple circular ribs formed in the outer skirt protruding radially outwardly from the outer skirt and frame.

FIG. 9A is a detail view of a portion of an outer skirt for a prosthetic heart valve showing a method for forming a radially outwardly protruding rib by extending a suture in an alternating pattern between apertures in two adjacent, circumferentially extending rows of apertures.

FIG. 9B is a detail view of the portion of the outer skirt of FIG. 9A with the radially outwardly protruding rib formed in the outer skirt.

FIG. 9C is another detail view of the portion of the outer skirt of FIG. 9A with the radially outwardly protruding rib formed in the outer skirt.

FIG. 10A is a detail view of a portion of an outer skirt for a prosthetic heart valve showing a method for forming a radially outwardly protruding rib by extending a suture in an alternating pattern between apertures in three adjacent, circumferentially extending rows of apertures.

FIG. 10B is a detail view of the portion of the outer skirt of FIG. 10A with the radially outwardly protruding rib formed in the outer skirt.

FIG. 11A is a detail view of a portion of an outer skirt for a prosthetic heart valve showing a method for forming a radially outwardly protruding rib by extending a suture in an alternating pattern between apertures in two adjacent, circumferentially extending rows of apertures, through one layer of a folded portion of the outer skirt.

FIG. 11B is a detail view of the portion of the outer skirt of FIG. 11A with the radially outwardly protruding rib formed in the outer skirt.

FIG. 12A is a detail view of a portion of an outer skirt for a prosthetic heart valve showing a method for forming a radially outwardly protruding rib by extending a suture in an alternating pattern between apertures in two adjacent, circumferentially extending rows of apertures, through both layers of a folded portion of the outer skirt.

FIG. 12B is a detail view of the portion of the outer skirt of FIG. 12A with the radially outwardly protruding rib formed in the outer skirt.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

As introduced above, most expandable prosthetic heart valves comprise a cylindrical metal frame or stent and prosthetic leaflets mounted inside the frame. These valves can also include one or more coverings (or skirts) spanning a circumference of the frame, on an inner or outer surface of the frame. These coverings can be configured to establish a seal with the native tissue when the prosthetic heart valve is radially expanded at the implantation site (and thus may be referred to as sealing members). However, the native tissue can have an irregular shape while the frame of the prosthetic heart valve is generally cylindrical. As a result, gaps can be formed between the prosthetic heart valve and native heart valve when the prosthetic heart valve is implanted within the native heart valve, even when coverings are included on the prosthetic heart valve. Accordingly, a need exists for improved coverings for prosthetic heart valves which can better fills gaps between the native tissue and the prosthetic heart valve.

Described herein are outer skirts or sealing members having one or more sealing members that protrude radially outward from a base surface of the outer skirt and the frame and that are configured to improve sealing of the prosthetic heart valve against the native tissue at an implantation site. As a result, paravalvular leakage can be reduced.

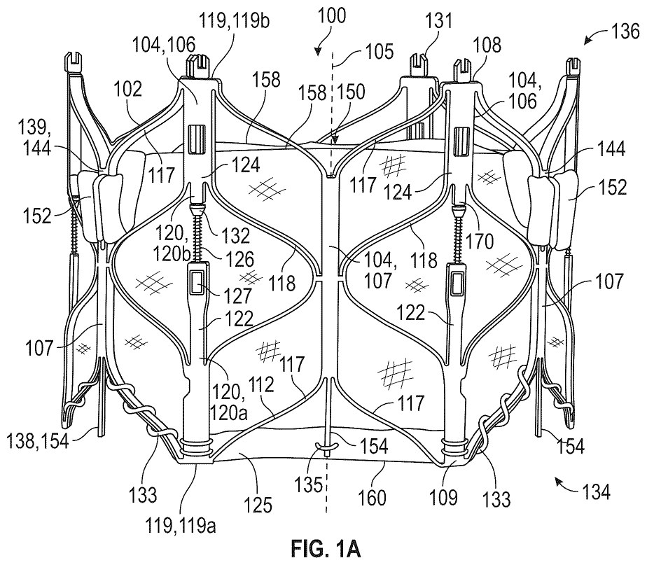

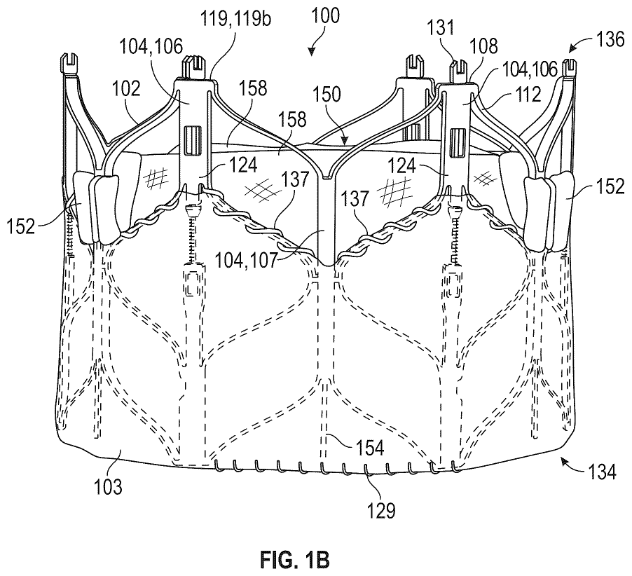

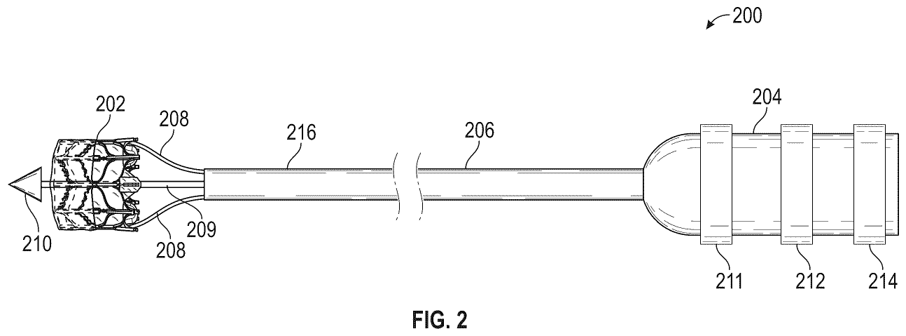

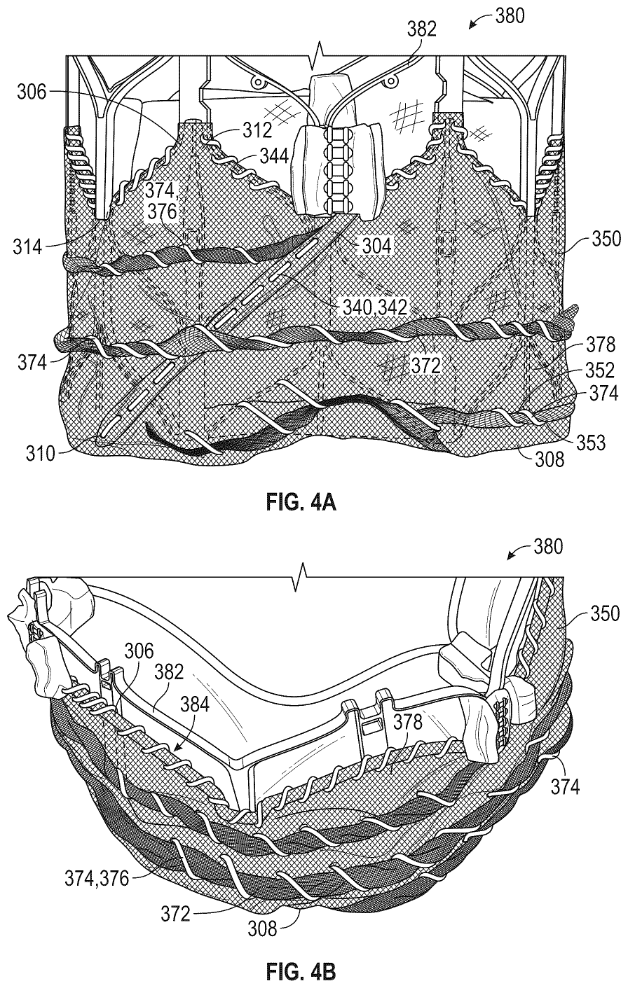

FIGS. 1A-1B illustrate an exemplary prosthetic device (e.g., prosthetic heart valve) that can be advanced through a patient's vasculature, such as to a native heart valve, by a delivery apparatus, such as the exemplary delivery apparatus shown in FIG. 2. The prosthetic heart valve can include an outer covering or skirt disposed around an outer surface of the frame of the prosthetic heart valve. The outer skirt can comprise one or more sealing members that protrude radially outward from a base surface of the outer skirt and the frame and that are configured to improve sealing of the prosthetic heart valve against native tissue at an implantation site. Various examples of protruding sealing members for outer skirts and methods of forming such protruding sealing members are shown in FIGS. 4A-12B.

### Examples of the Disclosed Technology

FIGS. 1A-1B show an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-1B illustrate an example of a prosthetic valve 100 (which also may be referred to herein as “prosthetic heart valve 100”) having a frame 102. FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 (which can also be referred to herein as a covering or sealing member) mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In other examples, as shown in FIG. 1A, the leaflets 158 can be sutured directly to the frame 102 or to a reinforcing member 125 (also referred to as a reinforcing skirt or connecting skirt) in the form of a strip of material (e.g., a fabric strip) which is then sutured to the frame 102, along the scallop line via stitches (e.g., whip stitches) 133.

The inner and outer skirts and the connecting skirt 125 can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102 comprises and inflow end 109, an outflow end 108, and a plurality of axially extending posts 104. The axial direction of the frame 102 is indicated by a longitudinal axis 105, which extends from the inflow end 109 to the outflow end 108 (FIG. 1A). Some of the posts 104 can be arranged in pairs of axially aligned first and second struts or posts 122, 124 (FIG. 1A). An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In other examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (i.e., each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in other examples.

The struts 112 can form and/or define a plurality of cells (i.e., openings) in the frame 102. For example, the struts 112 can at least partially form and/or define a plurality of first cells 117 and a plurality of second cells 118 that extend circumferentially around the frame 102. As illustrated in FIGS. 1A, each second cell 118 can be disposed within one of the first cells 117.

As illustrated in FIGS. 1A and 1B, the struts 112 of frame 102 can comprise a curved shape. Each first cell 117 can have an axially-extending hexagonal shape including first and second apices 119 (e.g., an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (e.g., distal apex 120a and proximal apex 120b) (FIG. 1A). In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap (those with actuators 126 can be referred to as actuator posts or actuator struts). Each first post 122 (i.e., the lower post shown in FIG. 1A) can extend axially from the inflow end 109 of the prosthetic valve 100 toward the second post 124, and the second post 124 (i.e., the upper post shown in FIGS. 1A and 1B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122 (FIG. 1A). The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (e.g., by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In other examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In other examples, not all pairs of posts 122, 124 need be expansion and locking mechanisms (i.e., actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap separating the posts 122, 124, and thereby radially compressing or radially expanding the prosthetic valve 100, respectively. Thus, the gap between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (e.g., the delivery apparatus 200 of FIG. 2, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (e.g., in the form of a nut, washer or flange) disposed thereon (FIG. 1A). Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (e.g., clockwise) can cause corresponding axial movement of the first and second posts 122, 124 toward one another, thereby radially expanding the frame 102, while rotation of the threaded rod 126 in an opposite second direction causes corresponding axial movement of the first and second posts 122, 124 away from one another, thereby radially compressing the frame. When the threaded rod 126 is rotated in the first direction, the head portion 131 of the rod 126 bears against an adjacent surface of the frame (e.g., an outflow apex 119b), while the nut 127 and the first post 122 travel proximally along the threaded rod 126 toward the second post 124, thereby radially expanding the frame. As the frame 102 moves from a compressed configuration to an expanded configuration, the gap between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIG. 1A). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (e.g., counterclockwise) causing the stopper 132 to push against (i.e., provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In other examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIG. 1A, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening therein.

The commissure opening (which can also be referred to herein as a “commissure window”) can extend radially through a thickness of the support post 107 and can be configured to accept a portion of a valvular structure 150 (e.g., a commissure 152) to couple the valvular structure 150 to the frame 102. For example, each commissure 152 can be mounted to a respective commissure support structure 144, such as by inserting a pair of commissure tabs of adjacent leaflets 158 through the commissure opening and suturing the commissure tabs to each other and/or the commissure support structure 144. In some examples, the commissure opening can be fully enclosed by the support post 107 such that a portion of the valvular structure 150 can be slid radially through the commissure opening, from an interior to an exterior of the frame 102, during assembly.

The frame 102 can comprise any number of support posts 107, any number of which can be configured as commissure support structures 144. For example, the frame 102 can comprise six support posts 107, three of which are configured as commissure support structures 144. However, in other examples, the frame 102 can comprise more or less than six support posts 107 and/or more or less than three commissure support structures 144.

The inflow end portion 138 of each support post 107 can comprise an extension 154 (show as a cantilevered strut in FIG. 1A) that extends toward the inflow end 109 of the frame 102. In some examples, the extension 154 can extend such that an inflow edge of the extension 154 aligns with or substantially aligns with the inflow end 109 of the frame 102. In use, the extension 154 can prevent or mitigate portions of an outer skirt 103 from extending radially inwardly and thereby prevent or mitigate any obstruction of flow through the frame 102 caused by the outer skirt 103. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures in the extensions 154.

As an example, each extension 154 can have an aperture or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (e.g., Nitinol, stainless steel or a cobalt-chromium alloy), such as in the form of a tube. The plurality of cells can be formed by removing portions (e.g., via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material (e.g., stainless stee, etc.). When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

Suitable plastically-expandable materials that can be used to form the frames disclosed herein (e.g., the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In other examples, the frame 102 can be formed from a self-expandable material (e.g., Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. Referring to FIG. 2, it illustrates an exemplary delivery apparatus 200 for delivering a prosthetic device or valve 202 (e.g., prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (e.g., actuator or threaded rod 126) on the prosthetic valve. For example, three actuator assemblies 208 can be provided for a prosthetic valve having three actuators. In other examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (e.g., threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (e.g., knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (e.g., clockwise) can retract the sheath 216 proximally relative to the prosthetic valve 202 and rotation of the first knob 211 in a second direction (e.g., counter-clockwise) can advance the sheath 216 distally. In other examples, the first knob 211 can be actuated by sliding or moving the first knob 211 axially, such as pulling and/or pushing the knob. In other examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (e.g., clockwise) can radially expand the prosthetic valve 202 and rotation of the second knob 212 in a second direction (e.g., counter-clockwise) can radially collapse the prosthetic valve 202. In other examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (e.g., threaded rod). Once the actuator assemblies 208 are uncoupled from the prosthetic valve 202, the delivery apparatus 200 can be removed from the patient, leaving just the prosthetic valve 202 in the patient.

Additional details on mechanically expandable prosthetic devices (e.g., valves) and actuator assemblies for delivery apparatus for mechanically expandable prosthetic devices can be found in International Application No. PCT/US2021/052745, which is incorporated by reference herein.

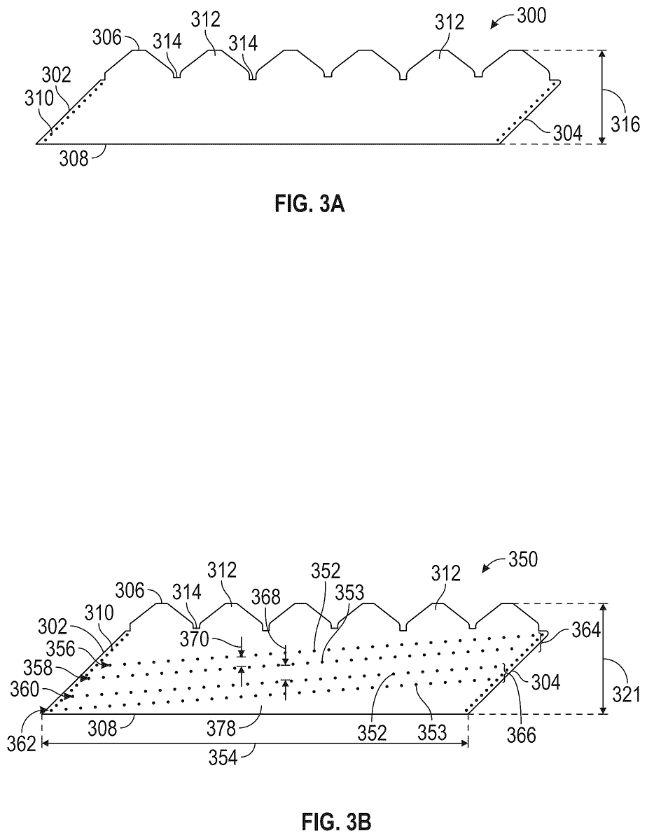

FIG. 3A shows an exemplary outer skirt 300 for a prosthetic device, such as the prosthetic valve 100 of FIGS. 1A and 1B, in a flattened configuration. The outer skirt 300, as well as the other outer skirts disclosed herein, can be used in a mechanically expandable prosthetic valve (e.g., valve 100 of FIGS. 1A and 1B), a balloon-expandable prosthetic valve, and/or a self-expandable prosthetic valve. Additional details on balloon expandable prosthetic valves can be found in U.S. Pat. No. 9,393,110, U.S. Provisional Application Nos. 63/178,416, filed Apr. 22, 2021, 63/194,830, filed May 28, 2021, and 63/279,096, filed Nov. 13, 2021, all of which are incorporated by reference herein.

The outer skirt 300 can be wrapped around and mounted to an outer surface of a frame of a prosthetic device (a radially outward facing surface relative to a central longitudinal axis of the prosthetic device), thereby transitioning to an annular configuration (e.g., as shown in FIG. 1B or 4A).

As shown in FIG. 3A, the outer skirt 300 can comprise opposing first and second edge portions 302, 304 (which can also be referred to as short edges or edge portions) which each extend between an outflow edge portion 306 and an inflow edge portion 308 of the outer skirt 300. The first and second edge portions 302, 304 can be non-perpendicular to the inflow edge portion 308. For example, the first and second edge portions 302, 304 can extend at angles of about 45 degrees (or in a range of 40 to 50 degrees) relative to the inflow edge portion 308. Therefore, an overall general shape of the outer skirt 300 can be that of a rhomboid or parallelogram.

The first and second edge portions 302, 304 can each comprise a plurality of apertures 310 extending therethrough. Thus, when the outer skirt 300 is converted into its annular configuration (e.g., when mounted around a prosthetic device, as shown in FIG. 4A for example), the first and second edge portions 302, 304 can overlap one another with their respective apertures 310 overlapping as well. A suture can then be used to form a plurality of stitches in and in-and-out pattern through the overlapping apertures 310 (e.g., stitches 340 shown in FIG. 4A), thereby securing the first and second edge portions 302, 304 together and forming the annular configuration of the outer skirt 300. As shown in FIG. 4A, due to the angled shape of the overlapping first and second edge portions 302, 304, the stitches 340 form a diagonally extending suture line 342.

The outflow edge portion 306 (which is disposed closer to the outflow end of the prosthetic device than the inflow edge portion 308 when arranged around the prosthetic device, e.g., as shown in FIG. 4A) can be formed with a plurality of projections 312 that define an undulating shape that generally follows the shape or contour of the struts of the frame to which they are secured (e.g., the struts 112 of the frame that are connected to the second posts 124 in FIGS. 1A and 1B, or the struts 344 shown in FIG. 4A).

In alternate examples, the outflow edge portion 306 can have a different shape than shown in FIG. 3A, such as straight (e.g., parallel to the inflow edge portion 308) or a different non-straight shape that does not follow the shape or contour of the struts of the frame to which they are secured.

In some examples, the outer skirt 300 can also be formed with slots 314 (or recesses) to facilitate attachment of the skirt to the frame. For example, each slot 314 can be arranged between two adjacent projections 312. In some examples, the slots 314 can be shaped and dimensioned such that the slots 314 fit against and/or can be wrapped or secured around the support posts of the frame (e.g., support posts 107 shown in FIG. 1B).

When the outer skirt 300 is arranged around an outer surface of the frame of the prosthetic device (e.g., as shown in FIG. 1B with outer skirt 103) sutures can be used to secure the outflow edge portion 306 of the outer skirt 300 to the struts of the frame (e.g., stitches 137 shown in FIG. 1B). In some examples, additional sutures can be used to secure the inflow edge portion 308 of the outer skirt 300 to the frame and/or leaflets of the prosthetic device (e.g., sutures 129 shown in FIG. 1B).

The outer skirt 300 and other skirts or covering described herein can comprise various synthetic materials, including fabrics (e.g., polyethylene terephthalate fabric (PET fabric) or ultra high molecular weight polyethylene (UHMWPE) fabric)), polytetrafluoroethylene (PTFE), thermoplastic polyurethane (TPU), a hybrid material comprising one or more fabric or polymeric materials (e.g., PET coated in TPU), or natural tissue (e.g., pericardial tissue). In some examples, the outer skirt 300 can be formed of a relatively flat fabric which allows for a relatively small crimp profile when the prosthetic device is in a radially compressed configuration. However, such a configuration may not allow the outer skirt 300 to efficiently conform to the native tissue and/or fill gaps between the native tissue and the implanted prosthetic device in a way that achieves enhanced paravalvular leakage sealing.

Thus, it may be desirable to provide an outer skirt, such as the outer skirt 300, with one or more sealing members that protrude radially outward from a base portion of the outer skirt and the frame of the prosthetic device, thereby providing increased paravalvular leakage sealing at the implantation site. For example, the outer skirt can be provided with a plurality of rows of apertures that are configured to receive one or more sutures (or other fibrous or polymeric tying members) therethrough, and upon tightening of the one or more sutures extending through the apertures, one or more radially protruding ribs or steps can be formed in the outer skirt. Various examples of creating such radially protruding ribs with the material of the outer skirt are described below with reference to FIGS. 3B-12B.

It should be noted that the radially protruding ribs described herein are described as being formed by tightened sutures extending through adjacent rows of apertures in an outer skirt. However, in other examples, the radially protruding ribs described herein can be formed by tightened sutures extending through the outer skirt along adjacent circumferentially extending paths in the outer skirt. In this way, instead of extending through rows of pre-formed apertures in the outer skirt, the stitches of the sutures described below can extend through selected locations in the outer skirt that form the adjacent circumferentially extending rows described below. In some examples, the selected locations for the stitches of the suture(s) can be indicated by markers (such as ink), a suture guide plate placed against the outer skirt, or the like. In still other examples, the stitches described below that form the radially protruding ribs upon their tightening within the outer skirt can be threaded through the outer skirt by free hand or eye, thereby forming the circumferentially extending stitches described below. In some examples, the free-hand stitches or stitches extending through the rows of markers can form apertures (e.g., formed by the needle puncturing the fabric of the outer skirt) in the locations of the apertures shown in FIGS. 3B-12B.

FIG. 3B shows another exemplary outer skirt 350 for a prosthetic device in a flattened configuration. The outer skirt 350 can be similar to the outer skirt 300 of FIG. 3A, but the outer skirt 350 comprises a plurality of rows of apertures 352, 353 that extend across a length 354 of the outer skirt 350, between the first and second edge portions 302, 304 (where the length 354 turns into a circumference of the outer skirt 350 when the outer skirt 350 is disposed around a prosthetic device or valve, such as the prosthetic valve 380 shown in FIGS. 4A and 4B).

For example, as shown in FIG. 3B, the outer skirt 350 comprises a first row 356 of first or upper apertures 352 and a second row 358 of second or lower apertures 353 which are grouped into a first pair of rows of apertures 364. The outer skirt 350 further comprises a third row 360 of first or upper apertures 352 and a fourth row 362 of second or lower apertures 353 which are grouped into a second pair of rows of apertures 366. Each of the first row 356, second row 358, third row 360, and fourth row 362 can comprise a plurality of apertures 352 or 353 that are spaced apart from one another within the row and extend at an angle (e.g., a non-zero angle) across the length 354 of the outer skirt 300, relative to the inflow edge portion 308. As discussed further below with reference to FIGS. 4A and 4B, this angling of the rows of apertures can create a rib 372 that spirals around a circumference of the outer skirt 350 in its annular configuration when arranged around an outer surface of a frame 382 of the prosthetic valve 380.

In some examples, a first spacing 368 between the first pair of rows of apertures 364 and the second pair of rows of apertures 366 (the spacing between the second row of apertures 358 and the third row of apertures 360) can be larger than a second spacing 370 between the rows of apertures within the same pair (e.g., the second spacing 370 between the first row 356 and the second row 358 and the second spacing 370 between the third row 360 and the fourth row 362). The first spacing 368 and the second spacing 370 can be selected based on a specified width of the resulting rib 372 and/or a specified number of revolutions or turns of the rib 372 around the circumference of the outer skirt 350 (when disposed around the frame 382 of the prosthetic valve 380, as shown in FIGS. 4A and 4B).

In some examples, an axial height 321 of the outer skirt 350 (before forming the sealing ribs) can be longer than an axial height of a portion of the frame to which the outer skirt 350 is to be coupled. For example, the axial height 321 can be longer than an axial height 316 of the outer skirt 300 which does not include apertures for forming one or more scaling ribs. In this way, the axial height 321 can provide additional skirt material (in the axial direction) to accommodate the rib 372 formed from the folded portion of the outer skirt 350.

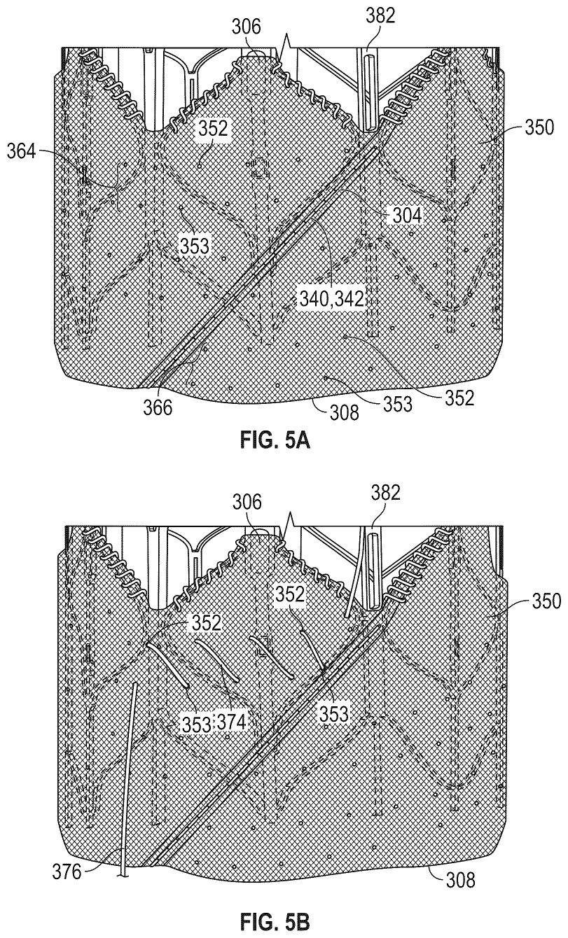

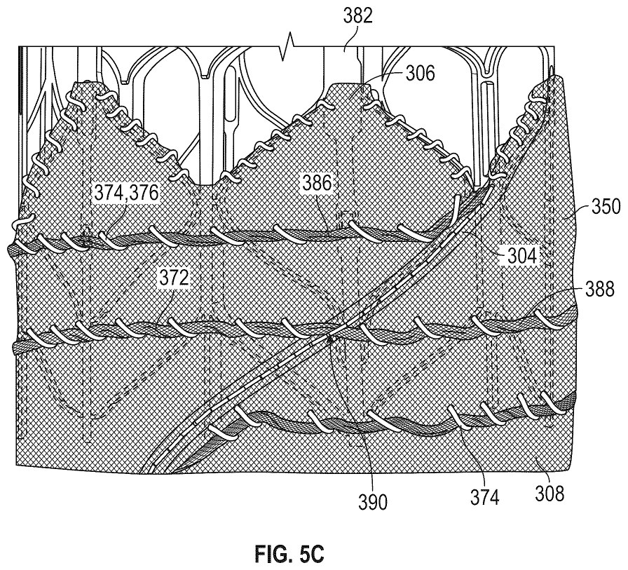

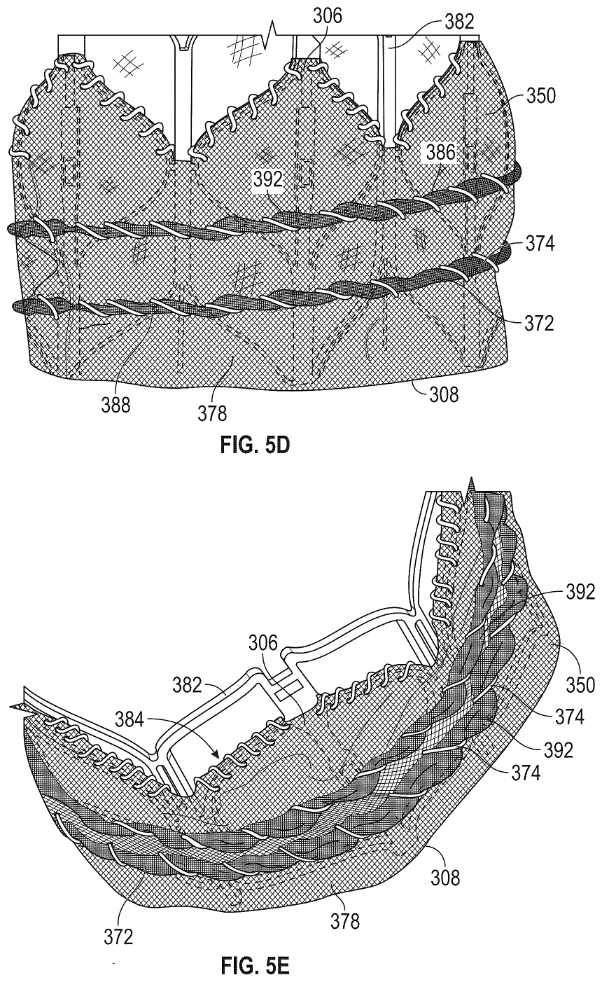

FIGS. 4A-4B show the outer skirt 350 disposed around and secured to the outer surface of the frame 382 of the prosthetic valve 380 (with leaflets arranged inside the frame 382), with the rib 372 already formed. FIGS. 5A-5E show the outer skirt 350 secured to the frame 382 (without leaflets inside the frame) and a series of illustrations depicting a method of forming the rib 372 in the outer skirt 350, while the outer skirt 350 is disposed around the frame 382.

When the outer skirt 350 is wrapped around an outer surface of the frame 382, the first and second edge portions 302, 304 are arranged such that they overlap one another and a suture can then be threaded in and in-and-out pattern through the overlapping apertures 310, thereby forming the diagonally extending suture line 342 of stitches 340 from the inflow edge portion 308 to one of the slots 314 proximate to the outflow edge portion 306 (FIGS. 4A and 5A). As a result, the first and second edge portions 302, 304 are secured together around the frame 382.

The prosthetic valve 380 (and its frame 382) can be similar to the prosthetic valve 100 of FIGS. 1A and 1B, with leaflets (e.g., leaflets 158) secured to an inner surface of the frame 382. In this way, the leaflets can be arranged on an inside of the frame 382 while the outer skirt 350 is arranged on an outside of the frame. As described above with reference to FIG. 1A, in some examples, an additional inner skirt can be disposed on an inner surface of the frame, between the leaflets and the inner surface of the frame.

When the outer skirt 350 is in its annular configuration, the apertures 352, 353 of the second pair of rows of apertures 366 that are disposed adjacent the first edge portion 302 can be continuous with the apertures 352, 353 of the first pair of rows of apertures 364 that are disposed adjacent the second edge portion 304 (FIGS. 5A-5B).

To form the rib 372 shown in FIGS. 4A and 4B, a suture 376 can be alternately passed in a zig-zagging pattern between the upper apertures 352 and lower apertures 353 in the first pair of rows of apertures 364 along the entire length of the first pair of rows 364, and then between upper apertures 352 and the lower apertures 353 in the second pair of rows of apertures 366 along the entire length of the second pair of rows 366, thereby forming a plurality of stitches 374 (FIG. 5B). In some examples, the stitches 374 can be angled (at a non-zero angle) relative to the inflow edge portion 308.

In some examples, the suture 376 can be passed through either the first pair of rows of apertures 364 or the second pair of rows of apertures 366 when the outer skirt 350 is still in its flattened configuration (FIG. 3B). However, the outer skirt 350 should then be placed in its annular configuration (or cylindrical shape, e.g., by arranging the outer skirt 350 around the frame 382) to allow the same suture 376 to continuously extend through the remainder of the apertures 352, 353 in the other one of the first pair of rows of apertures 364 or the second pair of rows of apertures 366.

After passing the suture 376 through all apertures 352, 353, it is tightened (for example, by pulling on its ends) to move the upper apertures 352 and lower apertures 353 through which it extends, toward each other, thereby folding the material of the outer skirt 350 caught in between the upper apertures 352 and lower apertures 353 (FIG. 5C) of each pair of rows 364, 366. This folding of the skirt material results in the formation of a radially outwardly protruding rib 372 (which can also be referred to as a “step” or “projection”), as shown in FIGS. 4A, 4B, and 5C-5E. The skirt material between the upper and lower apertures 352, 353 of the first pair of rows 364 forms a first portion or section of the rib 372 and the skirt material between the upper and lower apertures 352, 353 of the second pair of rows 366 forms a second portion or section of the rib 372.

As introduced above, due to the angled orientation of the rows of apertures 356, 358, 360, 362 (FIG. 3B), in the illustrated example, the rib 372 formed in the outer skirt 350 is a continuous rib that continuously spirals around the circumference of the outer skirt 350 from the inflow edge portion 308 toward the outflow edge portion 306 (FIGS. 4A-5E). FIGS. 5C-5E present different views of the outer skirt 350 secured around the frame 382 and show the continuous spiraling of the rib 372 around the circumference of the frame 382.

In other examples, multiple discontinuous ribs can be created in the outer skirt 350 by creating gaps between the pairs of rows of apertures in the outer skirt 350 and utilizing multiple sutures 376.

The two pairs of rows of apertures 364, 366 of the outer skirt 350 (FIG. 3B) result in a spiraling rib 372 having two revolutions around the frame 382, as shown in FIGS. 4A, 4B and 5C-5E. For example, as shown in FIG. 5C, a first revolution 386 of the rib 372 extends from the overlapping first and second edge portions 302, 304 proximate to the outflow edge portion 306, and around the circumference of the outer skirt 350 to the overlapping first and second edge portions 302, 304 at a location 390 between the outflow edge portion 306 and the inflow edge portion 308. A second revolution 388 of the rib 372 extends from the overlapping first and second edge portions 302, 304 at the location 390 between the outflow edge portion 306 and the inflow edge portion 308, and around the circumference of the outer skirt 350 to the overlapping first and second edge portions 302, 304 proximate to the inflow edge portion 308.

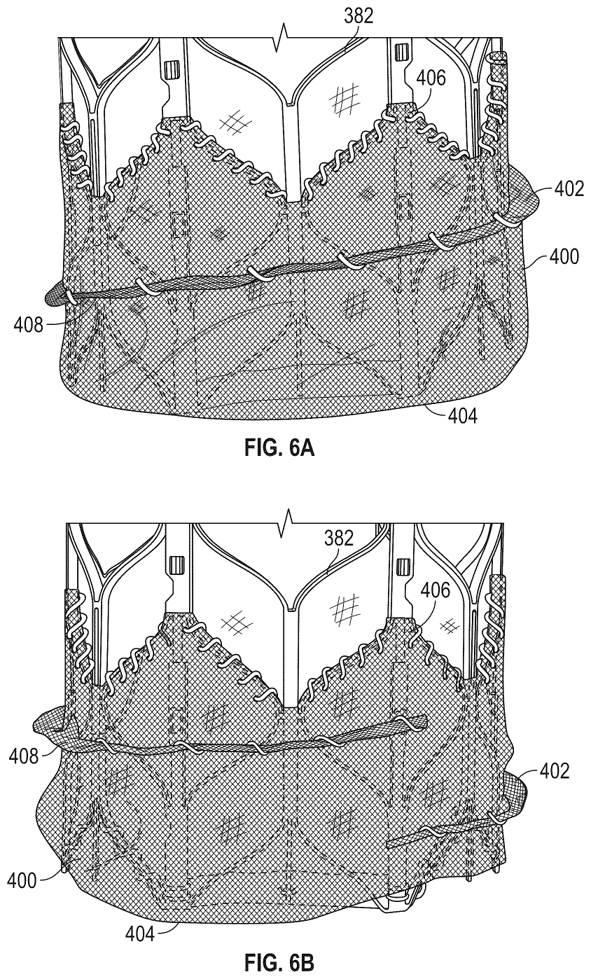

In other examples, the rows of apertures of the outer skirt 350 can be configured such that more or less that two revolutions of the spiraling rib 372 are formed (e.g., a single revolution, 1.5 revolutions, three revolutions, or the like). For example, as shown in FIGS. 6A and 6B, an outer skirt 400 can comprise a continuous radially outwardly protruding rib 402 that spirals around the outer skirt 400 between an inflow edge portion 404 and an outflow edge portion 406 of the outer skirt 400. The rib 402 spirals around the outer skirt 400 for approximately a single revolution 408. As used herein, a “revolution” can refer to a path of 360 degrees. As shown in FIG. 6B, the ends of the rib 402 can be spaced axially from each other along the height of the skirt 350.

The outer skirt 400 can be similar to the outer skirt 350 of FIG. 3B. However, instead of having two pairs of rows of apertures 364, 366, the outer skirt 400 can comprise only a single pair of rows of apertures that extends along a length of the outer skirt 400, at an angle relative to the inflow edge portion 404. Said another way, the outer skirt 400 can comprise a first row of first or upper apertures and a second row of second or lower apertures, the first and second rows extending parallel to one another and at an angle along a length of the outer skirt 400 from the inflow edge portion 404 to a location proximate the outflow edge portion 406 (e.g., proximate to one of the slots 314).

It should be noted that in FIGS. 6A and 6B and several of the additional examples herein (e.g., FIGS. 5A-5E, 7A-7B, 8A-8C), the figures show the outer skirt arranged around the frame, prior to the inflow edge portion of the outer skirt being secured to the frame. Thus, some of these figures may show the inflow edge portion, or portions thereof, distal or proximal to the inflow end of the frame. However, after securing the inflow edge portion of the skirt to the inflow end of the frame, the inflow edge portion of the skirt can extend circumferentially along the inflow end of the frame (e.g., as shown in FIGS. 4A-4B and 1B).

Returning to FIGS. 4A-5E, the two revolutions 386 and 388 of the rib 372 of the outer skirt 350 can provide a larger radially protruding surface area for increased sealing with the native anatomy (e.g., native valve annulus), as compared to outer skirts having a rib with only one revolution around the prosthetic valve.

As shown in FIGS. 4A, 4B, and 5C-5E, the formed rib 372 extends radially outward from a base surface 378 of the outer skirt 350. The base surface 378 can be an outer surface of the outer skirt 350 which faces radially outward relative to a central longitudinal axis of the frame 382. An inner surface 384 of the outer skirt 350 (FIGS. 4B and 5E) that is opposite the base surface 378 can be disposed against (e.g., flush against) the outer surfaces of the struts of the frame 382. Thus, the rib 372 protrudes radially outward and away from the base surface 378 and the frame 382 (and thus can be referred to herein as a protruding rib or sealing member).

In some examples, the rib 372 can twist at least partially between adjacent stitches 374 due to the tightening of the suture 376 (FIGS. 5C-5E). In some examples, a plurality of petals or rib portions 392 are formed along the rib 372, each rib portion 392 defined between two adjacent stitches 374 (FIGS. 5D and 5E). Each rib portion 392 extends radially outward from the base surface 378 and away from the frame 382. In some examples, one or more rib portions 392 can assume a slightly different shape or extend radially outward by a different amount than one or more other rib portions 392.

The plurality of rib portions 392 can provide a non-uniform shape to the rib 372. This can provide better sealing against the native anatomy (e.g., a wall of an atrium or valve annulus) since the native anatomy may have an uneven surface with a plurality of non-uniform depressions. For example, each petal or rib portion 392 can be pressed into a respective depression in the native anatomy where the prosthetic valve is implanted. In this way, each rib portion 392 can conform independently, apart from the other rib portions 392 of the rib 372, to a respective portion of the native anatomy.

In general, the spiral shape of the rib 372 can better conform to an irregular shape of the surrounding native anatomy (as compared to a circular rib, as explained further below) and can spread easier in an axial direction, thereby reducing a crimp profile of the prosthetic valve when in a radially compressed configuration.

The rib 372 and the similar radially protruding sealing members described herein can be formed from a relatively flat or two-dimensional fabric (as shown in FIG. 3B), without requiring the fabric to be pre-formed with any special 3D features or undergo heat-setting or other thermal manipulations. More specifically, the sealing members and ribs described herein can be formed from a relatively flat skirt with one or more sutures extending through pre-formed apertures in the skirt. As such, the ribs or radially protruding sealing members can be easily and efficiently formed during assembly of a prosthetic heart valve (or similar prosthetic device). Further, while the rib 372 or other sealing members can be formed in an outer surface of the skirt (outer or base surface 378), the inner surface 384 can remain relatively flat without protruding into an interior of the frame 382, thereby reducing interaction between the leaflets and the outer skirt. Further, by forming the rib 372 or other sealing members described herein from a same material as a remainder of the outer skirt, a method of fabricating the skirt and assembling the prosthetic device can be simplified.

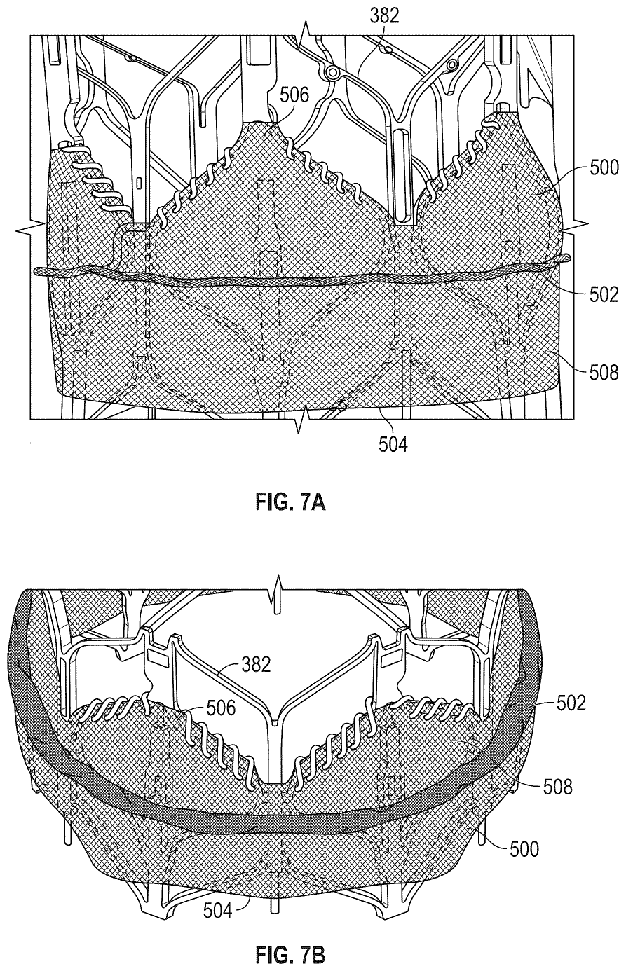

In other examples, the rib or multiple ribs formed in the outer skirt can be circular (ring-like or forming a closed circle) and extend around the circumference of the outer skirt at a relatively fixed axial position, without spiraling. For example, FIGS. 7A and 7B show an exemplary outer skirt 500 secured to and around an outside of the frame 382 for a prosthetic valve. The outer skirt 500 can be similar to the outer skirt 350, except it includes a single, radially protruding circular rib 502 (instead of a rib that spirals around the frame 382). The circular rib 502 extends around the circumference of the outer skirt 500 and frame 382 at a relatively fixed axial position that is disposed between an inflow edge portion 504 and outflow edge portion 506 of the outer skirt 500, thereby forming a ring around the outer skirt 500.

As best seen in FIG. 7B, the circular rib 502 protrudes (or extends) radially outward and away from a base surface 508 of the outer skirt 500 and the frame 382.

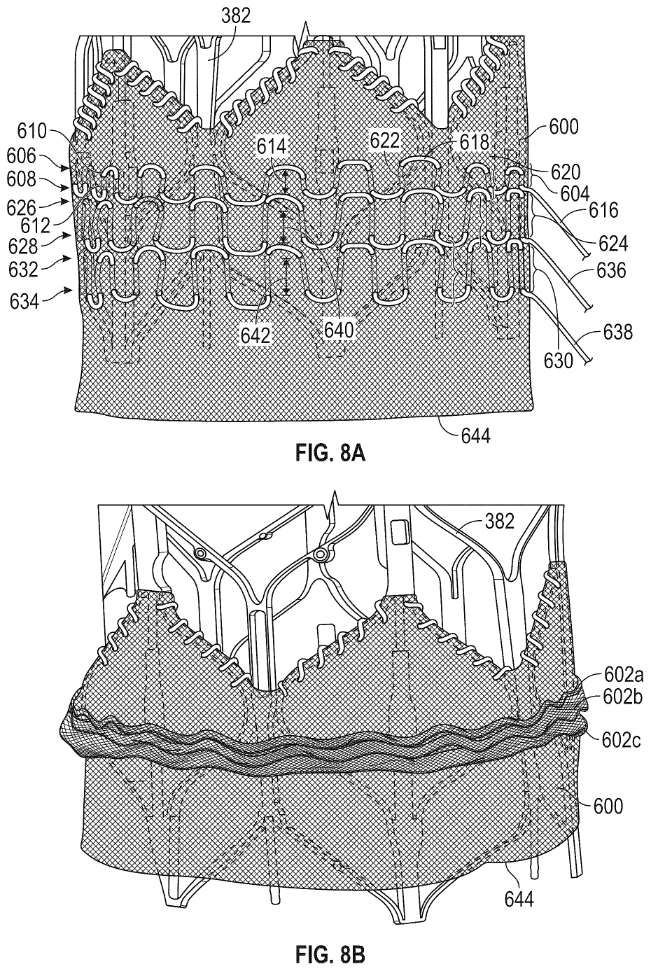

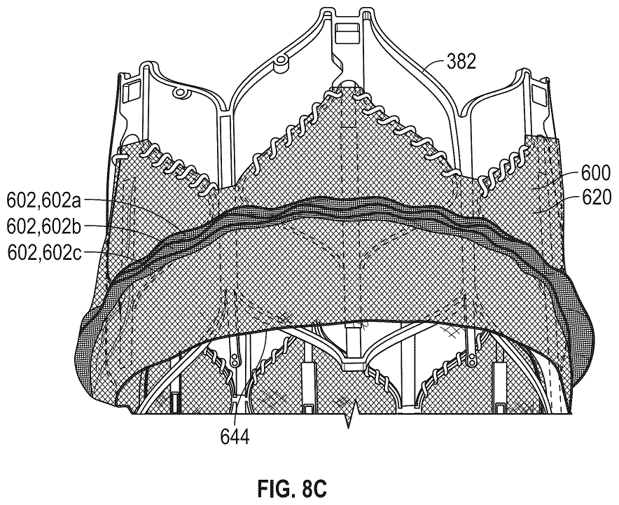

In some examples, the outer skirt 500 can comprise a plurality of circular ribs 502 that are spaced apart from one another in an axial direction (relative to a central longitudinal axis of the frame 382) along the height of the skirt. For example, FIGS. 8A-8C show another exemplary outer skirt 600 disposed around and attached to an outer surface of the frame 382 or the prosthetic valve. The outer skirt 600 comprises a plurality of circular ribs 602 (three are shown in FIGS. 8B and 8C, though more or less than three circular ribs are possible) which each extend circumferentially around the outer skirt 600 (e.g., each can extend for one full revolution around the outer skirt 600 and frame 382).

The circular ribs 602 can be formed by passing sutures (in some examples, one suture for each rib 602) through multiple pairs of horizontally or circumferentially extending (e.g., without spiraling or angling) rows of apertures, such as three pairs of rows of apertures (FIG. 8A). For example, FIG. 8A shows a first pair of rows of apertures 604 including a first row 606 of first (or upper) apertures 610 and a second row 608 of second (or lower) apertures 612. The first row 606 and the second row 608 can be arranged adjacent to one another. In some examples, the first row 606 and the second row 608 are parallel to one another. Further, the first row 606 and the second row 608 can be separated from one another in the axial direction by a first spacing 614.

A first circular rib 602a (FIG. 8B) can be formed by extending a first suture 616 through the second apertures 612 of the second row 608 and the first apertures 610 of the first row 606 in an alternating U-shaped pattern such that stitches 618 are formed on an outer surface 620 of the outer skirt 600, each stitch 618 extending between two adjacent first apertures 610 or two adjacent second apertures 612. The suture 616 can also form a plurality of axially extending stitches 622 on an inner surface of the outer skirt 600 (which is opposite the outer surface 620 and disposed against the frame 382) extending between and interconnecting adjacent stitches 618. The axially extending stitches 622 are shown in FIG. 8A with light lines to denote them being located behind the outer skirt 600 in the view of FIG. 8A (on the inner surface of the outer skirt 600). Tightening the first suture 616 pulls the skirt material between the first and second rows 606 and 608 closer together to form the first rib 602a.

Similarly, the outer skirt 600 can further include a second pair of rows of apertures 624 including a third row 626 of first apertures 610 and a fourth row 628 of second apertures 612, and a third pair of rows of apertures 630 including a fifth row 632 of first apertures 610 and a sixth row 634 of second apertures 612. A second circular rib 602b (FIG. 8B) can be formed by extending a second suture 636 through the first apertures 610 and the second apertures 612 of the second pair of rows of apertures 624 in the alternating U-shaped pattern and tightening the suture 636. A third circular rib 602c (FIG. 8B) can be formed by extending a third suture 638 through the first apertures 610 and the second apertures 612 of the third pair of rows of apertures 630 in the alternating U-shaped pattern (FIG. 8A) and tightening the suture 638.

In some examples, the spacing between the adjacent rows of each pair of rows of apertures 604, 624, and 630 can be the same.

In other examples, the spacing between the adjacent rows of each pair of rows of apertures 604, 624, and 630 can be different. For example, as shown in FIG. 8A, a second spacing 640 between the third row 626 and the fourth row 628 can be larger than the first spacing 614, and a third spacing 642 between the fifth row 632 and the sixth row 634 can be larger than the second spacing 640.

The first spacing 614, second spacing 640, and third spacing 642 can be selected based on a desired width (in the radial direction) or amount that the rib projects radially outward from the outer surface 620 of the outer skirt 600.

When the sutures 616, 636, and 638 are tightened, the first circular rib 602a, second circular rib 602b, and third circular rib 602c are formed adjacent one another in the axial direction (FIGS. 8B and 8C). Such an arrangement can result in increased PVL sealing when the prosthetic valve comprising the outer skirt 600 is implanted at the native anatomy (e.g., a native heart valve annulus).

In some examples, as shown in FIGS. 8B and 8C, the circular ribs 602a, 602b, 602c are disposed approximately parallel to an inflow edge portion 644 of the outer skirt 600.

As shown in FIGS. 8B and 8C the three circular ribs 602a, 602b, 602c are spaced slightly apart from one another in the axial direction. In some examples, the axial spacing between the pairs of rows of apertures can be increased, such that the three circular ribs 602a, 602b, 602c are spaced further apart from one another in the axial direction.

Further, in other examples, the outer skirt 600 can comprise more or less than three circular ribs 602 (e.g., two, four, five, or the like).

In some examples, the ribs (or sealing members or folds of the skirt) formed in the outer skirts described herein can extend axially (vertically) along the height of the outer skirt (e.g., between an inflow edge portion 308 and the outflow edge portion 306 of the outer skirt 350), rather than circumferentially around the outer skirt.

In other examples, an outer skirt can comprise ribs (or folds) that extend both axially and circumferentially. For example, the outer skirt can comprise one or more of the circular or spiraling ribs described above and also one or more axial ribs that extend in the axial direction along a height of the outer skirt. In some examples the axial ribs or folds can overlap or intersect with one or more of the circular or spiraling ribs or folds of the outer skirt.

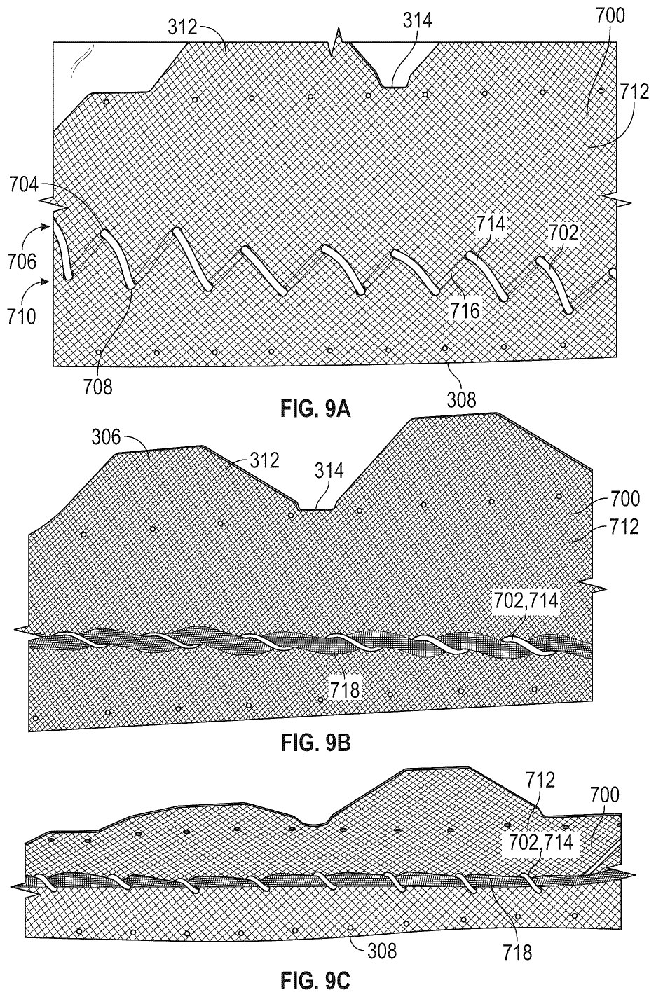

FIGS. 9A-12B illustrate various stitching methods for creating the circular or spiral ribs (or sealing members) described herein.

Turning first to FIGS. 9A-9C, an exemplary method for rib formation in an exemplary skirt 700 that is configured to be positioned around and secured to an outer surface of a frame of a prosthetic valve (such as frame 382) is shown. The skirt 700 can be similar to the other skirts described herein, such as outer skirt 350 shown in FIG. 3B.

As shown in FIG. 9A, the rib formation method can include passing a suture 702 between first (upper) apertures 704 in a circumferentially extending first row of apertures 706 and second (lower) apertures 708 in a circumferentially extending second row of apertures 710 in a zig-zag pattern. Passing the suture 702 in the zig-zag pattern can include passing the suture 702 in an alternating manner from a second aperture 708 to a first aperture 704 over a first side 712 of the skirt 700, thereby forming a first stitch 714 across the first side 712, and then from the first aperture 704 to a subsequent second aperture 708 along an opposite, second side of the skirt 700 (the side arranged “behind” the first side 712 in the view of FIG. 9A), thereby forming a second stitch 716 over the second side of the skirt 700. This can be repeated along a length of the skirt 700 until the suture 702 has been passed through all apertures 704, 708 of the first row 706 and the second row 710.

When the suture 702 is tightened, a radially protruding rib 718 can be formed (FIGS. 9B and 9C). The rib 718 can protrude radially outward from the first side 712 (or surface), which can be an outer surface of the skirt 700 which faces radially outward and away from a frame of a prosthetic device when the skirt 700 is secured around an outside of the frame.

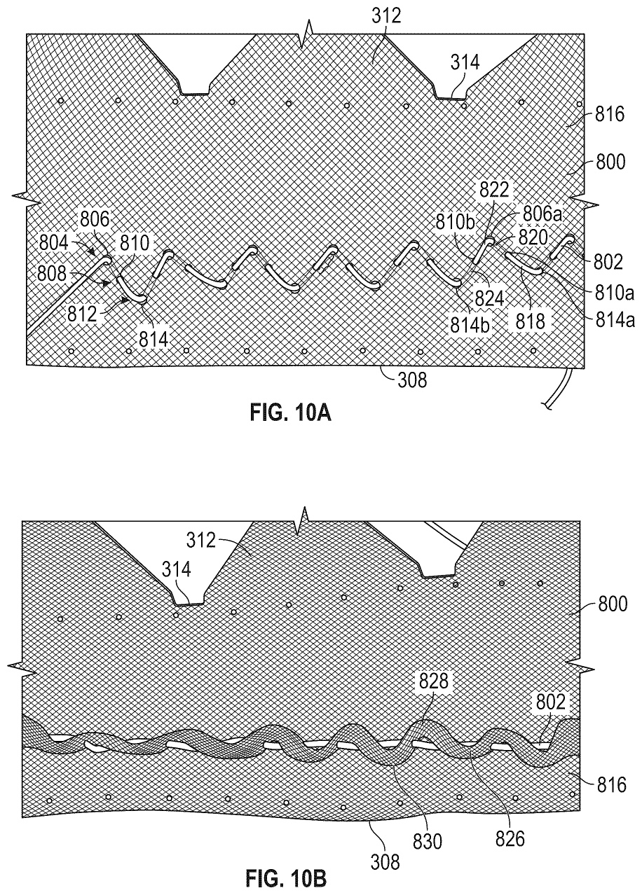

FIGS. 10A and 10B show another exemplary method for rib formation in an exemplary skirt 800 that is configured to be positioned around and secured to an outer surface of a frame of a prosthetic valve (such as frame 382) is shown. The skirt 800 can be similar to the other skirts described herein, such as outer skirt 350 shown in FIG. 3B.

As shown in FIG. 10A, the rib formation method can include passing a suture 802 between three adjacent rows of apertures in a zig-zag pattern. The three rows of apertures can include a first row 804 of first (upper) apertures 806, a second (middle) row 808 of second (middle) apertures 810, and a third (lower) row 812 of third (lower) apertures 814. For example, the method can include passing the suture 802 from a first (lower) aperture 814a to a second (middle) aperture 810a over a first side 816 of the skirt 800, thereby forming a first stitch 818, then from the second aperture 810a to a third (upper) aperture 806a over an opposite, second side of the skirt 800, thereby forming a second stitch 820 (FIG. 10A). The method can then include passing the suture 802 from the third aperture 806a to a subsequent second (middle) aperture 810b over the first side 816 again, thereby forming a third stitch 822, and then from the second aperture 810b to a subsequent first (lower) aperture 814b over the opposite second side, thereby forming a fourth stitch 824 (FIG. 10A). This can be repeated along a length of the skirt 800 until the suture 802 has been passed through all apertures 806, 810, 814 of the first row 804, the second row 808, and the third row 812.

When the suture 802 is tightened, a radially protruding rib 826 can be formed (FIG. 10B). The rib 826 can protrude radially outward from the first side 816 (or surface) of the skirt 800, which can be an outer surface of the skirt 800 which faces radially outward and away from a frame of a prosthetic device when the skirt 800 is secured around an outside of the frame.

The rib 826 formed by the method of FIGS. 10A and 10B can comprise first rib portions 828 disposed above (or on a first side of) the tightened suture 802 and second rib portions 830 disposed below (or on an opposite, second side of) the tightened suture 802 such that the rib 826 can form an undulating (e.g., sinusoidal) pattern along its length having a plurality of peaks and valleys along the length of the rib. In some examples, this can result in a rib 826 having a more irregular shape that can increase PVL sealing against the native anatomy after implantation of a prosthetic device around which the skirt 800 is secured.

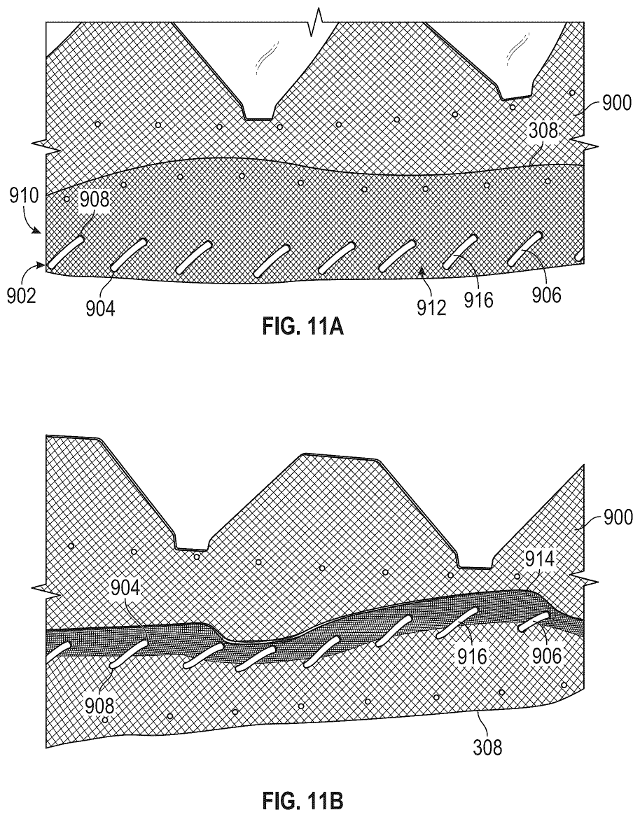

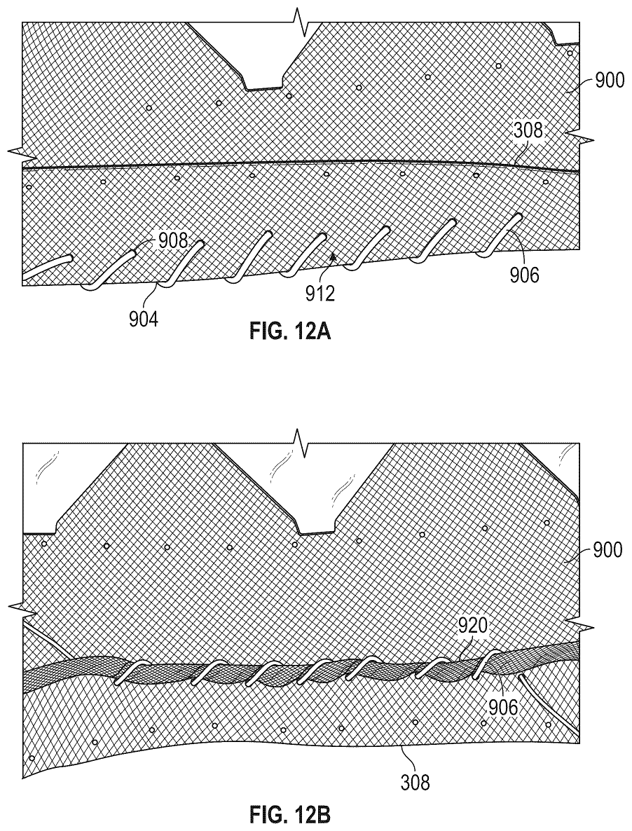

FIGS. 11A-12B show additional examples for rib formation in which a portion of a skirt 900 is folded over itself along a first row 902 of apertures 904. In the example of FIG. 11A, a suture 906 can be passed in a zig-zag pattern between the apertures 904 of the first row 902 and apertures 908 of an adjacent second row 910, through a single layer of the fold 912 (e.g., the top layer shown in FIG. 11A), thereby creating stitches 916. As a result, a radially outwardly protruding rib 914 is formed when the suture 906 is tightened and the skirt 900 is unfolded (FIG. 11B).

In another example, a rib 920 can be formed in the skirt 900 (FIG. 12B) by passing the suture 906 in a zig-zag pattern between the apertures 904 and apertures 908, through both layers of the fold 912 (FIG. 12A), tightening the suture 906, and then unfolding the skirt 900 (FIG. 12B).

The ribs or sealing members described herein can be referred to as “non-spreadable” due to their ability to remain in their folded form both when the prosthetic valve is in a radially compressed and radially expanded configuration. Thus, the ribs or sealing members described herein can remain in the folded state, even when the prosthetic valve is radially compressed.

In some examples, the ribs or sealing members (e.g., the folded portions of the skirt) described herein can be formed by using attachment means other than sutures (e.g., suture 376 shown in FIGS. 4A, 4B, and 5B-E) to secure the folded portions of skirt material to each other, such as an adhesive, ultrasonic welding, or fasteners.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame; a plurality of leaflets arranged within an interior of the frame; and an outer skirt disposed around an outer surface of the frame, wherein the outer skirt comprises at least one folded portion and stitches extending through the folded portion to form a radially protruding rib.

Example 2. The prosthetic heart valve of any example herein, particularly example 1, wherein an inner surface of the outer skirt is positioned against the outer surface of the frame, wherein a base surface of the outer skirt is a radially outward facing surface of the outer skirt that is disposed opposite the inner surface, and wherein the rib extends radially outward from the base surface and the frame.

Example 3. The prosthetic heart valve of any example herein, particularly either example 1 or example 2, wherein the annular frame comprises a plurality of interconnected struts, and wherein the outer skirt is positioned flush against outer surfaces of the plurality of interconnected struts.

Example 4. The prosthetic heart valve of any example herein, particularly any one of examples 1-3, wherein the rib extends between an inflow edge portion and an outflow edge portion of the outer skirt.

Example 5. The prosthetic heart valve of any example herein, particularly example 4, wherein the inflow edge portion of the outer skirt is secured to an inflow end of the frame, and wherein the outflow edge portion of the outer skirt is secured to struts of the frame that are arranged between the inflow end and an outflow end of the frame.

Example 6. The prosthetic heart valve of any example herein, particularly any one of examples 1-5, wherein the rib is a circumferentially extending rib that extends around an entire circumference of the outer skirt and the frame.

Example 7. The prosthetic heart valve of any example herein, particularly any one of examples 1-6, wherein the rib is a spiraling rib that continuously spirals around the outer skirt, between an inflow edge portion and an outflow edge portion of the outer skirt, for one or more revolutions.

Example 8. The prosthetic heart valve of any example herein, particularly example 7, wherein the spiraling rib continuously spirals around the outer skirt for two revolutions.

Example 9. The prosthetic heart valve of any example herein, particularly any one of examples 1-6, wherein the rib is a first rib, and wherein the outer skirt includes a second folded portion and stitches extending through the second folded portion to form a radially protruding second rib, and wherein each of the first rib and the second rib extend around an entire circumference of the outer skirt and form two radially protruding rings.

Example 10. The prosthetic heart valve of any example herein, particularly any one of examples 1-9, wherein the outer skirt comprises a first row of apertures and a second row of apertures that are parallel to each other and each extend around the circumference of the outer skirt, and wherein the rib is formed between the first and second rows of apertures by a tightened suture extending in an alternating pattern between adjacent apertures of the first row of apertures and the second row of apertures to form a plurality of spaced apart stitches that extend around the circumference of the outer skirt.

Example 11. The prosthetic heart valve of any example herein, particularly example 10, wherein the rib comprises a plurality of radially outwardly protruding rib portions, each rib portion defined between two adjacent stitches of the plurality of stitches.

Example 12. The prosthetic heart valve of any example herein, particularly either example 10 or example 11, wherein the first row of apertures and the second row of apertures extend around the circumference of the outer skirt at a non-zero angle relative to an inflow edge portion of the outer skirt.

Example 13. The prosthetic heart valve of any example herein, particularly any one of examples 10-12, wherein the outer skirt further comprises a third row of apertures and a fourth row of apertures that are parallel to each other and each extend around the circumference of the outer skirt, the third and fourth rows of apertures spaced axially apart from the first and second rows of apertures, wherein the third and fourth rows of apertures are continuous with the first and second rows of apertures, respectively, and wherein the rib is formed between the first and second rows of apertures and the third and fourth rows of apertures by the tightened suture extending in an alternating pattern between adjacent apertures of the first row of apertures and the second row of apertures and adjacent apertures of the third row of apertures and the fourth row of apertures.

Example 14. The prosthetic heart valve of any example herein, particularly example 13, wherein the rib is a single rib of the outer skirt that spirals around the circumference of the outer skirt for two revolutions.

Example 15. The prosthetic heart valve of any example herein, particularly any either example 10 or example 11, wherein the first row of apertures and the second row of apertures extend around the circumference of the outer skirt in parallel to an inflow edge portion of the outer skirt.

Example 16. The prosthetic heart valve of any example herein, particularly example 15, wherein the outer skirt further comprises a third row of apertures and a fourth row of apertures that are parallel to each other and each extend around the circumference of the outer skirt, the third and fourth rows of apertures spaced axially apart from the first and second rows of apertures, wherein the rib is a first rib that is formed between the first and second rows of apertures by a first tightened suture extending in an alternating pattern between adjacent apertures of the first row of apertures and the second row of apertures, and wherein a second rib is formed between the third and fourth rows of apertures by a second tightened suture extending in an alternating pattern between adjacent apertures of the third row of apertures and the fourth row of apertures.

Example 17. The prosthetic heart valve of any example herein, particularly example 16, wherein the first rib is spaced axially apart from the second rib, and wherein the first and second ribs are configured as annular ribs that each extend around an entirety of the circumference of the outer skirt.

Example 18. The prosthetic heart valve of any example herein, particularly any one of examples 1-17, wherein the outer skirt comprises a fabric.

Example 19. The prosthetic heart valve of any example herein, particularly any one of examples 1-18, wherein the outer skirt comprises an inflow edge portion, an outflow edge portion, and opposing first and second angled edge portions that extend between the inflow edge portion and the outflow edge portion, wherein the first and second angled edge portions angle at a non-zero angle relative to the inflow edge portion, and wherein the first and second angle edge portions overlap one another and are secured together such that the outer skirt forms an annular configuration around the outer surface of the frame.

Example 20. The prosthetic heart valve of any example herein, particularly example 19, wherein the inflow edge portion is straight, and wherein the outflow edge portion has an undulating shape defined by a plurality of projections.

Example 21. A prosthetic heart valve comprising: an annular frame comprising a plurality of interconnected struts; and an outer skirt disposed around an outer surface of the frame, the outer skirt comprising: an inner surface disposed against the outer surface of the frame and an outer surface arranged opposite the inner surface; a circumferentially extending first row of first apertures and a circumferentially extending second row of second apertures; a plurality of stitches formed between the first and second rows, each stitch of the plurality of stitches extending between a respective one of the first apertures and a respective one of the second apertures; and a folded portion of the outer skirt between the first and second rows of apertures that forms a rib that protrudes radially outward from the outer surface of the outer skirt and away from the frame.

Example 22. The prosthetic heart valve of any example herein, particularly example 21, wherein the first row of first apertures and the second row of second apertures are disposed parallel to one another and extend at a non-zero angle along the circumference of the outer skirt relative to an inflow edge portion of the outer skirt that is secured to an inflow end of the frame.

Example 23. The prosthetic heart valve of any example herein, particularly either example 21 or example 22, wherein the rib spirals around the circumference of the outer skirt, from an inflow edge portion of the outer skirt toward an outflow edge portion of the outer skirt.

Example 24. The prosthetic heart valve of any example herein, particularly example 23, wherein the rib spirals around the circumference of the outer skirt for a single revolution.

Example 25. The prosthetic heart valve of any example herein, particularly example 23, wherein the outer skirt further comprises a circumferentially extending third row of first apertures and a circumferentially extending fourth row of second apertures, the third and fourth rows disposed parallel to one another and spaced axially apart from the first and second rows, wherein the plurality of stitches are further formed between the third and fourth rows, and wherein a first revolution of the rib is formed between the first and second rows and a second revolution of the rib is formed between the third and fourth rows.

Example 26. The prosthetic heart valve of any example herein, particularly example 21, wherein the first row of first apertures and the second row of second apertures extend around a circumference of the outer skirt in parallel to an inflow edge portion of the outer skirt that is secured to an inflow end of the frame.

Example 27. The prosthetic heart valve of any example herein, particularly example 26, wherein the rib forms a ring around an entire circumference of the outer skirt.

Example 28. The prosthetic heart valve of any example herein, particularly either example 26 or example 27, wherein the outer skirt further comprises a circumferentially extending third row of first apertures and a circumferentially extending fourth row of second apertures, the third and fourth rows disposed parallel to one another and spaced axially apart from the first and second rows, wherein the rib formed between the first and second rows is a first rib, and wherein a second rib is formed between the third and fourth rows, the second rib comprising a folded portion of the outer skirt that protrudes radially outward from the outer surface of the outer skirt and away from the frame and forms a ring around an entire circumference of the outer skirt, the first and second ribs spaced apart from one another in an axial direction.

Example 29. The prosthetic heart valve of any example herein, particularly any one of examples 21-28, wherein the plurality of stitches comprises alternating first stitches and second stitches, wherein the first stitches each extend between a respective first aperture and second aperture on the outer surface of the outer skirt, and wherein the second stitches each extend between a respective first aperture and second aperture on the inner surface of the outer skirt.

Example 30. The prosthetic heart valve of any example herein, particularly any one of examples 21-28, wherein the outer skirt further comprises a circumferentially extending third row of third apertures, wherein the plurality of stitches is formed between the first, second, and third rows, each stitch extending between a respective one of the third apertures and a respective one of the first apertures or second apertures.

Example 31. The prosthetic heart valve of any example herein, particularly example 30, wherein the plurality of stitches comprises alternating first stitches and second stitches, wherein the first stitches each extend between a respective third aperture and a respective first or second aperture on the outer surface of the outer skirt, and wherein the second stitches each extend between a respective third aperture and a respective first or second aperture on the inner surface of the outer skirt.

Example 32. The prosthetic heart valve of any example herein, particularly any one of examples 21-31, wherein the outer skirt comprises an inflow edge portion secured to an inflow end of the frame and an outflow edge portion secured to a first portion of the plurality of interconnected struts that are connected to a plurality of axially extending posts of the frame, the plurality of axially extending posts being further connected to a second portion of the plurality of interconnected struts that define an outflow end of the frame.

Example 33. The prosthetic heart valve of any example herein, particularly example 32, wherein the inflow edge portion is straight, and wherein the outflow edge portion has an undulating shape defined by a plurality of projections.

Example 34. The prosthetic heart valve of any example herein, particularly any one of examples 21-33, wherein the inner surface of the outer skirt is disposed flush against the outer surface of the frame.

Example 35. The prosthetic heart valve of any example herein, particularly any one of examples 21-34, further comprising a plurality of leaflets arranged within an interior of the frame, and wherein the plurality of interconnected struts is disposed between the plurality of leaflets and the outer skirt.

Example 36. A method of assembling a prosthetic heart valve, comprising: securing a fabric skirt around an outer surface of a frame of a prosthetic heart valve; extending a suture in an alternating pattern between first apertures of a first row of spaced apart first apertures in the skirt and second apertures of a second row of spaced apart second apertures in the skirt, the first and second rows extending circumferentially along the skirt and spaced apart from one another in an axial direction; and tightening the suture to form a protruding rib between the first and second rows, the protruding rib comprising a folded portion of the skirt that protrudes radially outward from a remainder of the skirt that is disposed against the frame.

Example 37. The method of any example herein, particularly example 36, wherein extending the suture in the alternating pattern includes passing the suture in a zig-zag pattern from a second aperture of the second apertures to a first aperture of the first apertures over a first side of the skirt and then from the first aperture to a subsequent second aperture over an opposite, second side of the skirt such that a plurality of spaced apart stitches are formed around a circumference of the skirt.

Example 38. The method of any example herein, particularly example 37, wherein the protruding rib protrudes radially outward from the first side of the skirt, and wherein the second side of the skirt is disposed flush against the outer surface of the frame.

Example 39. The method of any example herein, particularly any one of examples 36-38, wherein securing the skirt around the outer surface of the frame includes wrapping the skirt around the outer surface of the frame, overlapping first and second edge portions of the skirt which are angled relative to an inflow edge portion of the skirt, and securing together the overlapping first and second edge portions with an angled stitch line that extends between the inflow edge portion and an outflow edge portion of the skirt.

Example 40. The method of any example herein, particularly example 39, wherein securing the skirt around the outer surface of the frame further includes securing the inflow edge portion to an inflow end of the frame and securing the outflow edge portion to a plurality of angled struts extending around a circumference of the frame at a location disposed axially between the inflow end and an outflow end of the frame.

Example 41. The method of any example herein, particularly any one of examples 36-40, wherein the first row and the second row extend circumferentially along the skirt at a non-zero angle relative to an inflow edge portion of the skirt that is secured to an inflow end of the frame.

Example 42. The method of any example herein, particularly example 41, wherein the protruding rib spirals around a circumference of the skirt for one or more revolutions, from the inflow edge portion of the skirt toward an outflow edge portion of the skirt.

Example 43. The method of any example herein, particularly any one of examples 36-40, wherein the first row and the second row extend circumferentially along the skirt in parallel to an inflow edge portion of the skirt that is secured to an inflow end of the frame.

Example 44. The method of any example herein, particularly example 43, wherein the protruding rib forms a ring around an entire circumference of the skirt.

Example 45. The method of any example herein, particularly any one of examples 36-44, further comprising arranging a plurality of leaflets within an interior of the frame.

Example 46. A prosthetic heart valve comprising: an annular frame comprising a plurality of interconnected struts; a plurality of leaflets arranged within an interior of the frame; and an outer skirt disposed around an outer surface of the frame, the outer skirt comprising: an inflow edge portion secured to an inflow end of the frame; an outflow edge portion secured to a first portion of the plurality of interconnected struts; opposing inner and outer surfaces, wherein the inner surface faces and is disposed against the outer surface of the frame; and a sealing member that spirals around a circumference of the outer skirt, between the inflow edge portion and toward the outflow edge portion, wherein the sealing member comprises a folded portion of the outer skirt that extends radially outward from the outer surface of the outer skirt and away from the frame.

Example 47. The prosthetic heart valve of any example herein, particularly example 46, wherein the first portion of the plurality of interconnected struts are disposed adjacent to a second portion of the plurality of interconnected struts that form an outflow end of the frame.

Example 48. The prosthetic heart valve of any example herein, particularly either example 46 or example 47, wherein the sealing member spirals for two revolutions around the outer skirt.

Example 49. The prosthetic heart valve of example herein, particularly example 48, wherein the outer skirt further comprises a plurality of projections that form an undulating shape of the outflow edge portion, and wherein the sealing member spirals between the inflow edge portion and a slot formed between two adjacent projections of the plurality of projections.

Example 50. The prosthetic heart valve of any example herein, particularly any one of examples 46-49, wherein the folded portion of the outer skirt of the sealing member is formed by a plurality of tightened stitches that extend around the circumference of the outer skirt in an alternating pattern between adjacent apertures of a first row of apertures and a second row of apertures that are disposed in the outer skirt, and wherein the first row of apertures and the second row of apertures are parallel to one another and spiral around the circumference of the outer skirt.

Example 51. The prosthetic heart valve of any example herein, particularly example 50, wherein the plurality of tightened stitches comprise a first portion of stitches arranged over the outer surface of the outer skirt and a second portion of stitches arranged over the inner surface of the outer skirt, the first portion of stitches and the second portion of stitches alternating with one another around the circumference of the outer skirt.

Example 52. The prosthetic heart valve of any example herein, particularly either example 50 or example 51, wherein the plurality of tightened stitches form a plurality of rib portions that each extend circumferentially between two adjacent stitches of the plurality of stitches and radially outward from the outer surface of the outer skirt.

Example 53. The prosthetic heart valve of any example herein, particularly any one of examples 46-52, wherein the sealing member extends circumferentially around the outer skirt at an angle relative to the inflow edge portion of the outer skirt.

Example 54. A prosthetic heart valve comprising: a radially expandable and compressible annular frame; a plurality of leaflets arranged within an interior of the frame; and an outer skirt disposed around an outer surface of the frame, the outer skirt comprising one or more folded portions forming one or more radially outwardly protruding ribs that remain folded when the prosthetic heart valve is in a radially compressed state and a radially expanded state.

Example 55. The prosthetic heart valve of any example herein, particularly example 54, wherein an inner surface of the outer skirt is positioned against the outer surface of the frame, and wherein the one or more ribs protrude radially outward from an outer surface of the outer skirt that is disposed opposite the inner surface of the outer skirt.

Example 56. The prosthetic heart valve of any example herein, particularly either example 54 or example 55, wherein the frame comprises a plurality of interconnected struts, and wherein the outer skirt is positioned flush against outer surfaces of the plurality of interconnected struts.

Example 57. The prosthetic heart valve of any example herein, particularly any one of examples 54-56, wherein the one or more ribs extend between an inflow edge portion and an outflow edge portion of the outer skirt.

Example 58. The prosthetic heart valve of any example herein, particularly example 57, wherein the inflow edge portion of the outer skirt is secured to an inflow end of the frame, and wherein the outflow edge portion of the outer skirt is secured to struts of the frame that are arranged between the inflow end and an outflow end of the frame.

Example 59. The prosthetic heart valve of any example herein, particularly any one of examples 54-58, wherein each rib of the one or more ribs extends around an entire circumference of the outer skirt and the frame.

Example 60. The prosthetic heart valve of any example herein, particularly any one of examples 54-59, wherein the one or more ribs includes a spiraling rib that continuously spirals around the outer skirt, between an inflow edge portion and an outflow edge portion of the outer skirt, for one or more revolutions.

Example 61. The prosthetic heart valve of any example herein, particularly example 60, wherein the spiraling rib continuously spirals around the outer skirt for two revolutions.

Example 62. The prosthetic heart valve of any example herein, particularly any one of examples 54-59, wherein the one or more ribs includes one or more ribs that each extend around an entire circumference of the outer skirt and form one or more radially protruding rings.

Example 63. The prosthetic heart valve of any example herein, particularly any one of examples 54-62, wherein the outer skirt comprises a first row of apertures and a second row of apertures that are parallel to each other and each extend circumferentially along the outer skirt, and wherein the one or more ribs are formed between the first and second rows of apertures by a tightened suture extending in an alternating pattern between adjacent apertures of the first row of apertures and the second row of apertures to form a plurality of spaced apart stitches that extend circumferentially along the outer skirt.

Example 64. The prosthetic heart valve of any example herein, particularly example 63, wherein the one or more ribs comprises a plurality of radially outwardly protruding rib portions, each rib portion defined between two adjacent stitches of the plurality of stitches.

Example 65. The prosthetic heart valve of example herein, particularly either examples 63 or example 64, wherein the first row of apertures and the second row of apertures extend circumferentially along the outer skirt at a non-zero angle relative to an inflow edge portion of the outer skirt.

Example 66. The prosthetic heart valve of any example herein, particularly any one of examples 63-65, wherein the outer skirt further comprises a third row of apertures and a fourth row of apertures that are parallel to each other and each extend circumferentially along the outer skirt, the third and fourth rows of apertures spaced axially apart from the first and second rows of apertures, wherein the third and fourth rows of apertures are continuous with the first and second rows of apertures, respectively, and wherein the one or more ribs are formed between the first and second rows of apertures and the third and fourth rows of apertures by the tightened suture extending in an alternating pattern between adjacent apertures of the first row of apertures and the second row of apertures and adjacent apertures of the third row of apertures and the fourth row of apertures.

Example 67. The prosthetic heart valve of any example herein, particularly example 66, wherein the one or more ribs comprises a single rib that spirals around the circumference of the outer skirt for two revolutions.

Example 68. The prosthetic heart valve of any example herein, particularly either example 63 or example 64, wherein the first row of apertures and the second row of apertures extend circumferentially along the outer skirt in parallel to an inflow edge portion of the outer skirt.

Example 69. The prosthetic heart valve of any example herein, particularly example 68, wherein the outer skirt further comprises a third row of apertures and a fourth row of apertures that are parallel to each other and each extend circumferentially along the outer skirt, the third and fourth rows of apertures spaced axially apart from the first and second rows of apertures, wherein a first rib of the one or more ribs is formed between the first and second rows of apertures by a first tightened suture extending in an alternating pattern between adjacent apertures of the first row of apertures and the second row of apertures, and wherein a second rib of the one or more ribs is formed between the third and fourth rows of apertures by a second tightened suture extending in an alternating pattern between adjacent apertures of the third row of apertures and the fourth row of apertures.

Example 70. The prosthetic heart valve of any example herein, particularly example 69, wherein the first rib is spaced axially apart from the second rib, and wherein the first and second ribs are configured as annular ribs that each extend around an entirety of the circumference of the outer skirt.

Example 71. The prosthetic heart valve of any example herein, particularly any one of examples 54-70, wherein the outer skirt comprises a fabric.

Example 72. The prosthetic heart valve of any example herein, particularly any one of examples 54-71, wherein the outer skirt comprises an inflow edge portion, an outflow edge portion, and opposing first and second angled edge portions that extend between the inflow edge portion and the outflow edge portion, wherein the first and second angled edge portions angle at a non-zero angle relative to the inflow edge portion, and wherein the first and second angle edge portions overlap one another and are secured together such that the outer skirt forms an annular configuration around the outer surface of the frame.

Example 73. The prosthetic heart valve of any example herein, particularly example 72, wherein the inflow edge portion is straight, and wherein the outflow edge portion has an undulating shape defined by a plurality of projections.

Example 74. A method comprising sterilizing the prosthetic heart valve, apparatus, and/or assembly of any example.

Example 75. A prosthetic heart valve of any one of examples 1-73, wherein the prosthetic heart valve is sterilized.

The features described herein with regard to any example can be combined with other features described in any one or more of the other examples, unless otherwise stated. For example, any one or more of the features of one skirt, covering, or sealing member can be combined with any one or more features of another skirt, covering, or sealing member. As another example, any one or more features of one prosthetic device can be combined with any one or more features of another prosthetic device.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame;
a plurality of leaflets arranged within an interior of the frame; and
an outer skirt disposed around an outer surface of the frame, wherein the outer skirt comprises at least one folded portion and stitches extending through the folded portion to form a radially protruding rib.

2. The prosthetic heart valve of claim 1, wherein an inner surface of the outer skirt is positioned against the outer surface of the frame, wherein a base surface of the outer skirt is a radially outward facing surface of the outer skirt that is disposed opposite the inner surface, and wherein the rib extends radially outward from the base surface and the frame.

3. The prosthetic heart valve of claim 1, wherein the annular frame comprises a plurality of interconnected struts, and wherein the outer skirt is positioned flush against outer surfaces of the plurality of interconnected struts.

4. The prosthetic heart valve of claim 1, wherein the rib extends between an inflow edge portion and an outflow edge portion of the outer skirt.

5. The prosthetic heart valve of claim 4, wherein the inflow edge portion of the outer skirt is secured to an inflow end of the frame, and wherein the outflow edge portion of the outer skirt is secured to struts of the frame that are arranged between the inflow end and an outflow end of the frame.

6. The prosthetic heart valve of claim 1, wherein the rib is a circumferentially extending rib that extends around an entire circumference of the outer skirt and the frame.

7. The prosthetic heart valve of claim 1, wherein the rib is a spiraling rib that continuously spirals around the outer skirt, between an inflow edge portion and an outflow edge portion of the outer skirt, for one or more revolutions.

8. The prosthetic heart valve of claim 7, wherein the spiraling rib continuously spirals around the outer skirt for two revolutions.

9. The prosthetic heart valve of claim 1, wherein the rib is a first rib, and wherein the outer skirt includes a second folded portion and stitches extending through the second folded portion to form a radially protruding second rib, and wherein each of the first rib and the second rib extend around an entire circumference of the outer skirt and form two radially protruding rings.

10. The prosthetic heart valve of claim 1, wherein the outer skirt comprises a first row of apertures and a second row of apertures that are parallel to each other and each extend around the circumference of the outer skirt, and wherein the rib is formed between the first and second rows of apertures by a tightened suture extending in an alternating pattern between adjacent apertures of the first row of apertures and the second row of apertures to form a plurality of spaced apart stitches that extend around the circumference of the outer skirt.

11. A prosthetic heart valve comprising:
an annular frame comprising a plurality of interconnected struts; and
an outer skirt disposed around an outer surface of the frame, the outer skirt comprising:
an inner surface disposed against the outer surface of the frame and an outer surface arranged opposite the inner surface;
a circumferentially extending first row of first apertures and a circumferentially extending second row of second apertures;
a plurality of stitches formed between the first and second rows, each stitch of the plurality of stitches extending between a respective one of the first apertures and a respective one of the second apertures; and
a folded portion of the outer skirt between the first and second rows of apertures that forms a rib that protrudes radially outward from the outer surface of the outer skirt and away from the frame.

12. The prosthetic heart valve of claim 11, wherein the first row of first apertures and the second row of second apertures are disposed parallel to one another and extend at a non-zero angle along the circumference of the outer skirt relative to an inflow edge portion of the outer skirt that is secured to an inflow end of the frame.

13. The prosthetic heart valve of claim 11, wherein the rib spirals around the circumference of the outer skirt, from an inflow edge portion of the outer skirt toward an outflow edge portion of the outer skirt.

14. The prosthetic heart valve of claim 13, wherein the outer skirt further comprises a circumferentially extending third row of first apertures and a circumferentially extending fourth row of second apertures, the third and fourth rows disposed parallel to one another and spaced axially apart from the first and second rows, wherein the plurality of stitches are further formed between the third and fourth rows, and wherein a first revolution of the rib is formed between the first and second rows and a second revolution of the rib is formed between the third and fourth rows.

15. The prosthetic heart valve of claim 11, wherein the first row of first apertures and the second row of second apertures extend around a circumference of the outer skirt in parallel to an inflow edge portion of the outer skirt that is secured to an inflow end of the frame.

16. The prosthetic heart valve of claim 15, wherein the rib forms a ring around an entire circumference of the outer skirt.

17. A prosthetic heart valve comprising:
a radially expandable and compressible annular frame;
a plurality of leaflets arranged within an interior of the frame; and
an outer skirt disposed around an outer surface of the frame, the outer skirt comprising one or more folded portions forming one or more radially outwardly protruding ribs that remain folded when the prosthetic heart valve is in a radially compressed state and a radially expanded state.

18. The prosthetic heart valve of claim 17, wherein an inner surface of the outer skirt is positioned against the outer surface of the frame, and wherein the one or more ribs protrude radially outward from an outer surface of the outer skirt that is disposed opposite the inner surface of the outer skirt.

19. The prosthetic heart valve of claim 17, wherein the one or more ribs extend between an inflow edge portion and an outflow edge portion of the outer skirt.

20. The prosthetic heart valve of claim 17, wherein the outer skirt comprises a first row of apertures and a second row of apertures that are parallel to each other and each extend circumferentially along the outer skirt, and wherein the one or more ribs are formed between the first and second rows of apertures by a tightened suture extending in an alternating pattern between adjacent apertures of the first row of apertures and the second row of apertures to form a plurality of spaced apart stitches that extend circumferentially along the outer skirt.
